(19) United States
(12) Patent Application Publication
(10) Pub. No.: US 2026/0260582 A1
(43) Pub. Date: Sep. 3, 2026

(54) CONSTRAINED SURGICAL TRAINING CONSOLE

(71) Applicant: Virtualisurg, Strasbourg (FR)

(72) Inventors: Tom Lorent Bourdo, Strasbourg (FR); Philippe Bertrand, Strasbourg (FR); Jean-Philippe Chevalier-Lancioni, Strasbourg (FR)

(21) Appl. No.: 19/483,142

(22) PCT Filed: May 13, 2024
(86) PCT No.: PCT/FR2024/050609

§ 371 Date: Nov. 10, 2025

(30) Foreign Application Priority Data
May 12, 2023 (FR) 2304768

(51) Int. Cl. G09B 23/28

(52) U.S. Cl. CPC G09B 23/285

(57) ABSTRACT

A surgical training console (10) having a base (12), a constraint module (16) having an opening (28) for passage of at least a portion of at least one surgical simulation tool (100) for manipulation by a user, a haptic arm (14) attached to the base (12), with a free end (26) configured to connect the surgical simulation tool. The haptic arm is mobile and makes it possible to define a working space (T) comprising all the positions that can be occupied by the free end. The base and the constraint module (16) are arranged to delimit an empty space defining a manipulation space (M), the manipulation space (M) being less than or equal to the working space (T), and the constraint module makes it possible to limit the positions that can be occupied by the free end of the haptic arm to those located within this manipulation space.

### TECHNICAL FIELD

The present invention relates to the field of training future surgeons. The present invention is therefore in the field of tools, methods and materials for education and teaching, in particular in the field of surgical training consoles.

### TECHNOLOGICAL BACKGROUND

To date, most surgical trainings are carried out in real conditions, on patients, through surgical companionship. This method requires significant human resources, presents high material constraints and can generate significant stress for the student which can lead to difficulties in concentration and/or memorization.

Alternatives exist, such as the Pelvitrainer EoSim SurgTrac® or certain sessions on animals. However, these trainings/methods are only accessible to a small number of surgical interns and have a number of obvious limitations: the Pelvitrainer is a simple box into which trocars and a camera are inserted with the possibility of performing sutures on inert materials such as foam. The animal model presents obvious problems in terms of training quality because the anatomical similarities/correlations with humans are limited. The animal model also poses more and more ethical problems.

The Pelvitrainer also poses ergonomic problems because the user cannot simulate the external gestures performed by surgeons during a surgical intervention, for example palpating the patient before inserting a surgical tool into their body, or stabilizing the intervention area by placing a hand on the patient externally, or to test the intervention area externally before operating, or simply to place your hand outside the intervention area for better operating comfort.

The aim of the present invention is therefore to provide a safe, practical, precise, realistic, easy-to-use and readily available training device, making it possible to increase the realism and ergonomics of use by allowing the user to use both hands to interact with the device, with or without a tool. The realism of surgical simulation depends largely on the possibility of applying realistic constraints to the movement of the manipulation tools held by the user. Applying these constraints is therefore a technical challenge.

### SUMMARY

The present invention therefore relates to a surgical training console (10), comprising:
a base,
a constraint module arranged at a distance from the base along an arrangement axis X, the constraint module having at least one opening enabling the passage of at least a part of at least one surgical simulation tool intended to be manipulated by a user,
a haptic arm secured to the base, the haptic arm having a free end configured to connect the at least one surgical simulation tool, the haptic arm being movable and making it possible to define a workspace comprising a set of positions that can be occupied by the free end,
a control unit connected to the haptic arm, the control unit being configured to generate a virtual reality and define parameters thereof so as to obtain, for the user, a simulation surgical intervention,
a display device connected to the control unit configured to display, to the user, a representation of a manipulation of the surgical simulation tool in the virtual reality generated by the control unit.

The application is characterized in that the parameters of the virtual reality comprise parameters for controlling the mobility of the haptic arm, the base and the constraint module are arranged to delimit an empty space defining a manipulation space (M), the manipulation space being less than or equal to the work space, and the constraint module enables to limit the positions which can be occupied by the free end of the haptic arm to those located within the manipulation space.

Thus, the solution allows to achieve the aforementioned objective. In particular, the presence of the constraint module makes it possible to technically achieve constraints on the last degrees of freedom of the haptic arm, in particular those far from the base, which are not easy to motorize without significantly weighing down the haptic arm.

The console according to the invention may comprise one or more of the following features, taken separately from one another or combined with one another:
the constraint module forms a tabletop,
the base and the constraint module are offset transversely along the arrangement axis X, so that the console has a stepped profile,
the arrangement distance between the base and the constraint module is variable,
the constraint module presents an inclination relative to the base,
this inclination is variable,
the base comprises an inclined foundation intended to receive a robot comprising the haptic arm,
the base and the constraint module are secured to each other in a reversible manner,
the at least one opening (28) has movable edges and a variable diameter,
the display device is associated with a mobile calibration tool and in that the console comprises a calibration module having an imprint intended to cooperate with the mobile calibration tool,
the calibration module can be reversibly attached to the base,
the display device is a virtual reality headset,
the haptic arm presents a resting position in which the free end is positioned facing the opening of the constraint module,

The present invention also relates to a surgical training kit, characterized in that it comprises a surgical training console according to the descriptions above and at least one surgical simulation tool.

This Surgical Training Kit can be characterized in that a virtual twin of the surgical simulation tool connected to the haptic arm is represented to the user by the display device.

### BRIEF DESCRIPTION OF THE FIGURES

The invention will be better understood, and other aims, details, characteristics and advantages thereof will appear more clearly on reading the detailed explanatory description which follows, of embodiments of the invention given by way of illustration, of purely illustrative and non-limiting examples, with reference to the appended drawings:

FIG. 1 is a perspective view of a surgical training console according to the present invention, with a surgical simulation tool,

FIG. 2a is a dissociated view of the surgical training console according to the embodiment of the preceding figure,

FIG. 2b is a detailed view of a calibration module,

FIG. 3 is a perspective view of a user interacting with the console according to the present invention, by means of a surgical simulation tool not represented by the display device,

FIG. 4 is a top view of the console according to the present invention.

FIGS. 5a and 5b are two perspective views of a user interacting with the console according to the present invention, by means of a surgical simulation tool represented by the display device.

FIG. 6 is a graphical visualization of the degrees of freedom of a haptic arm of the console according to the present invention.

### DETAILED DESCRIPTION

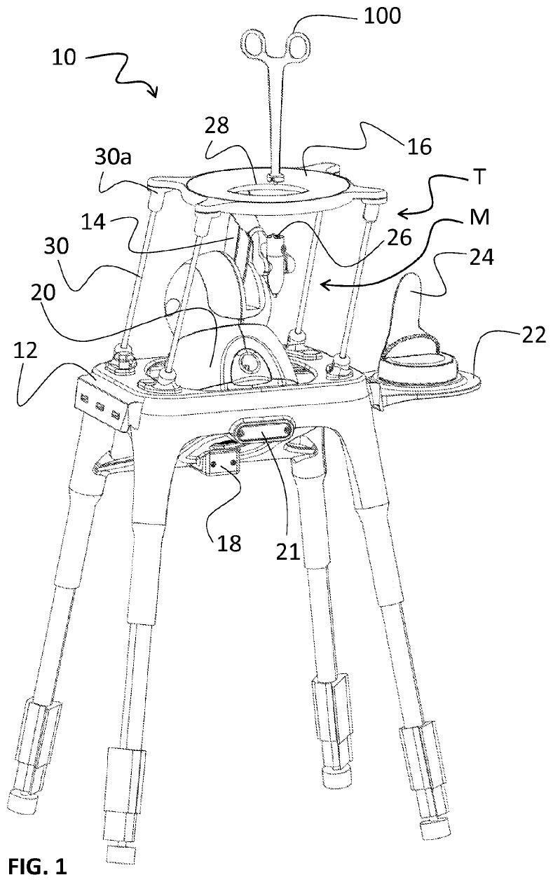

As visible in FIG. 1, the surgical training console 10 according to the present invention comprises:
a base 12,
a haptic arm 14 secured on the base 12,
a constraint module 16 arranged at a distance from the base 12 along an arrangement axis X,
a control unit 18 connected to the haptic arm 14, configured to generate a virtual reality and define parameters thereof so as to obtain, for a user, a simulation of a surgical intervention,
a display device (not shown) connected to the control unit 18.

The surgical training console 10 according to the present invention is used as a kit with at least one surgical simulation tool 100 intended to be connected to the haptic arm 14 (see below).

The display device is configured to display, to the user, a representation of a manipulation of the surgical simulation tool 100 in the virtual reality generated by the control unit 18. Preferably, it is a virtual reality headset.

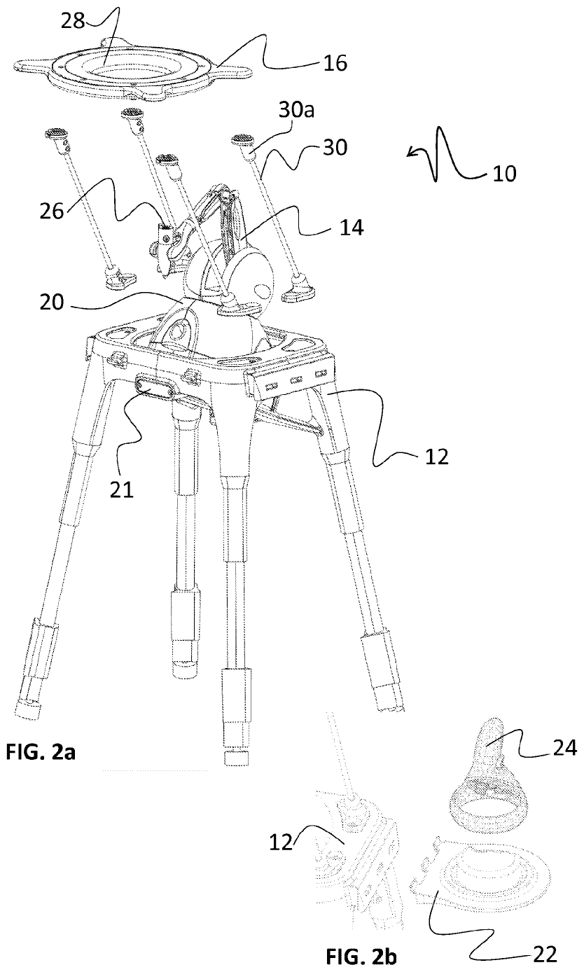

The base 12 presents, as visible in FIG. 2a, a cavity forming an inclined base intended to receive the body of a robot 20 comprising a haptic arm 14. According to the embodiment shown in FIGS. 1 and 2a, the base 12 has a substantially rectangular shape. This shape makes storage easier. Preferably, the dimensions of the base 12 of the console 10 are between 300 and 500 mm long and 200 to 500 mm wide. The base 12 presents a thickness between 150 and 300 mm.

In certain embodiments, the base 12 has at least one socket 21 connected, by an electrical circuit, to the control unit 18. This socket 21 makes it possible to electrically connect a surgical simulation tool 100 mechanically connected to the haptic arm 14 to the control unit 14.

As seen in FIGS. 1 and 2a, the base presents feet extending along the arrangement axis X. These feet have a variable and adjustable height, so as to adapt the height of the base 12 to the user and allow optimal comfort of use. The length of the feet can preferably vary between 100 and 500 mm.

As visible in FIG. 2b, the base 12 has at least one connection imprint intended to cooperate with a calibration module 22. It is thus possible to removably connect the calibration module 22 to the console 10. In the embodiment shown in FIG. 2a, the base 12 has a connection imprint on each of its faces, so as to be able to connect the calibration module 22 to different locations depending on the space required by the user to be able to carry out the simulation or, in other cases, to be able to connect several calibration modules 22 to the base 12.

The calibration module 22 is connected to the display device and is configured to align the virtual reality with the physical reality of the user manipulating the console 10 according to the present invention. In a manner known per se, the display device is associated with a mobile calibration tool 24 (see FIG. 2b). The mobile calibration tool 24 can take the form of a classic controller as for example illustrated in FIG. 2b but it can also take a different form. The calibration module 22 includes a complementary imprint of the mobile calibration tool 24. Thus, the calibration module 22 makes it possible to position the mobile calibration tool 24 associated with the display device at a known and fixed position relative to the base 12, and more particularly the haptic arm 14.

As the haptic arm 14 makes it possible to know the position and relative orientation of an object attached to it (see below), one obtains the position and orientation of this object relative to the mobile calibration tool 24. In the case where the display device is a mobile device configured to be worn by the user, the calibration module 22 further makes it possible to locate the user relatively to the base of the console 10. Furthermore, since the position of the mobile calibration tool 24 relatively to the display device is known, the position and orientation of the object connected to the haptic arm 14 relatively to the user wearing the display device are known.

As mentioned above, the haptic arm 14 is connected to the base 12 by means of the body of the robot 20 secured on said base 12. As can be seen on FIG. 2a, the haptic arm 14 presents, opposite the body of the robot 20, a free end 26 configured to connect at least one surgical simulation tool 100.

The haptic arm 14 presents a resting position when no surgical simulation tool 100 is connected to the free end 26. When in this resting position, the free end 26 is housed in a cavity or hole located in the body of the robot 20, preferably on its front face. In an alternative embodiment, the resting position of the haptic arm 14 may place the free end facing the opening 28 of the constraint module 16.

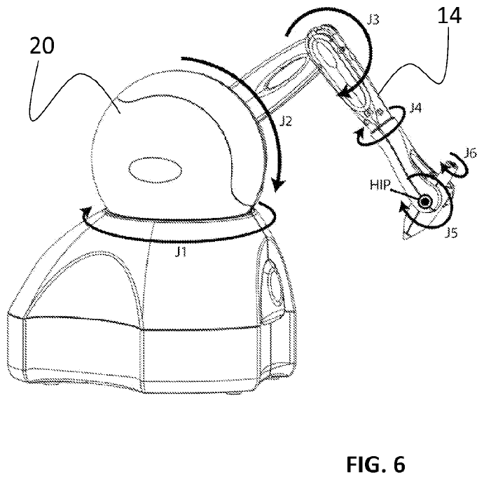

The haptic arm 14 is movable according to at least six degrees of freedom obtained by means of various elbows and rotating parts cooperating with each other so as to form joints J₁, J₂, J₃, J₄, J₅, J₆. More precisely, and as visible on FIG. 6, the first three joints (distal joints) are actuatable by the user while the last three joints (proximal joints) are passive.

In order to maximize the realism of the simulation, the surgical simulation tool 100, once connected to the haptic arm 14, must have its tip (or free end) positioned where the haptic feedback would occur in reality, i.e., at the haptic point of the haptic arm 14. This haptic point is designated as “HIP” on FIG. 6. The haptic arm 14 simulates force feedback related to the collision or interactions in the virtual world of the tip (or end) of the surgical simulation tool 100 manipulated with an element of the virtual environment. This is the point at which interactions and collisions are calculated to be able to simulate them without creating an uncomfortable and disturbing haptic lag or inconsistency for the user. Taking into account this haptic point HIP, makes it possible to simulate the penetration of a patient's body, for example by the needle of a syringe, by simulating the stress exerted by the patient's body on the needle.

All joints are tracked by position sensors to determine their respective angular positions and rotations, but not all of them benefit from haptic feedback. On FIG. 6, the joints that do not benefit from haptic feedback are referenced J₄, J₅ and J₆. Since the three distal joints have large dimensions, it is possible and easy to equip them with a motor that can limit their mobility if necessary.

This mobility of the haptic arm 14 makes it possible to define a workspace T comprising all of the positions that can be occupied by the free end 26 of the haptic arm 14. The mobility of the haptic arm 14 refers to the speed and ease with which a user can move the free end 26 of the haptic arm 14 from one position to another in the workspace T. This mobility is controlled by the control unit 18. The mobility of the haptic arm 14 is thus part of the parameters of the virtual reality generated by the control unit 18. Thus, the virtual reality parameters include all or part of the parameters for controlling the mobility of the haptic arm 14. This mobility can thus be adapted to a large number of different surgical simulations.

Depending on what the virtual reality generated by the control unit is intended to represent to the user, the mobility parameters of the haptic arm 14 vary and the haptic signal generated by the control unit and transmitted by the haptic arm 14 to the user also varies.

In the present application, the notion of “haptic signal” is understood as a signal actively generated by the console 10 according to the present invention. It should be distinguished from the notion of “tactile feedback” which is simple passive feedback, generated automatically by the human body in reaction to the manipulation of animate or inanimate objects.

The constraint module 16 is a physical constraint module. The constraint module 16 may, as visible in the figures, have a general tabletop shape. Depending on the simulation(s) chosen by the user, the constraint module 16 can have different shapes, more or less close to realistic anatomical shapes.

Regardless of its shape, the constraint module 16 has an opening 28 allowing the passage of at least part of at least one surgical simulation tool 100.

In some embodiments, the haptic arm 14 has a resting position in which the free end 26 is positioned inside the opening of the cover.

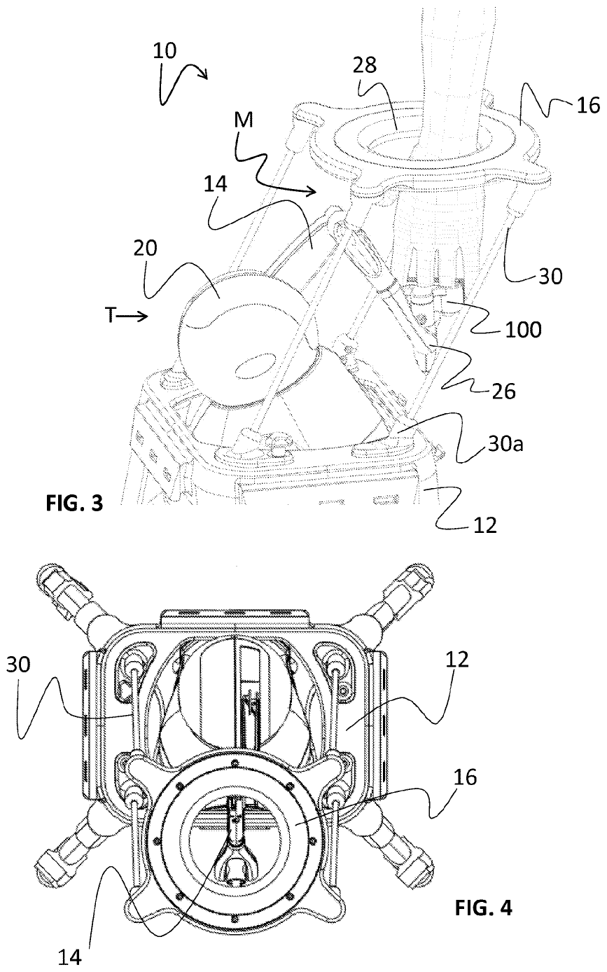

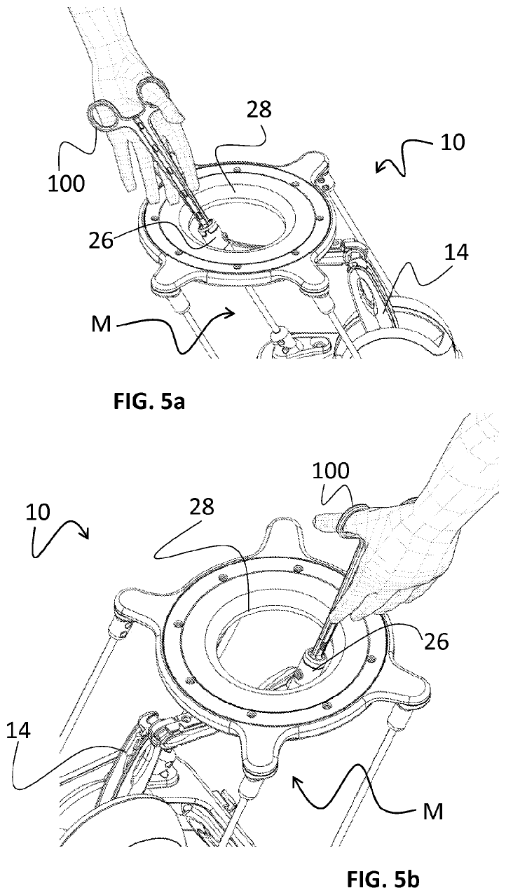

This opening 28 can be an opening with fixed edges or with moving, movable edges. Its diameter can therefore be fixed or variable. This opening 28, specifically, is a clever solution to the lack of haptic feedback from the last 3 degrees of freedom of the haptic arm 14. This opening 28 allows, for example, to better simulate surgery by helping the user to feel the walls of the opening created in the patient's body during a surgical intervention. In some embodiments illustrated on FIG. 3, the user can pass his entire hand through the opening 28, thus being able to create, for example, palpation simulations in the patient's body. In other embodiments illustrated on FIGS. 5a and 5b, the opening 28 does not allow the user to pass his hand through, but simply the end of the surgical simulation tool 100 manipulated by the user.

Preferably, the internal edges of the opening 28 are covered with silicone for realism purposes. In other embodiments, the aperture 28 may be directly connected to the control unit 18 to improve the accuracy of the simulation.

The constraint module 16 is removably secured to the base 12 by means of at least one spacer 30, preferably four spacers 30. The length of the spacers 30 allows the constraint module 16 to be arranged at a distance ranging between 200 and 400 mm from the base 12. Each spacer 30 preferably has a rod shape with a securing foot 30a at each end. Each securing foot 30a may be designed to cooperate with at least one clamping knob to maintain a strong physical connection between the constraint module 16 and the base 12. More precisely, the presence of magnets makes it possible to generate a magnetic lock-key type connection enabling to remove any possibility of removing the constraint module 16 with shear forces when using the console 10.

The securing between each securing foot 30a of each spacer 30 with the base 12 and/or the constraint module 16 can be reinforced by the presence of magnets. The set of spacers 30 thus allows easy assembly/disassembly of the console 10 and allows easy storage in a suitcase, for example. This reversible arrangement of base 12 and constraint module 16 also makes base 12 and constraint module 16 reusable, independently of each other, in other projects or other simulations.

Thus, the positioning of the at least one spacer 30 between the base 12 and the constraint module 16 makes it possible to arrange the base 12 and the constraint module 16 so as to delimit an empty space defining a manipulation space M.

In this application, the term “empty” is to be understood as an absence of physical obstacle. An empty space according to the present application is a space in which someone can freely move his hand (for example) throughout the volume of said space without being hindered by any element or object.

Once arranged, the constraint module 16 makes it possible to limit the positions that can be occupied by the free end 26 of the haptic arm 14. Due to its positioning relative to the base 12, the constraint module directly or indirectly restricts the mobility of the haptic arm 14. This mobility can be restricted directly if the constraint module 16 is arranged sufficiently close to the base 12 to physically prevent the free end 26 of the haptic arm from occupying each of the positions of the workspace T. This mobility can be restricted indirectly if the constraint module 16 is arranged so as to limit the movements of the user, thus preventing the user from positioning the free end 26 of the haptic arm 14 in each of the positions of the workspace T.

This constrained space is the manipulation space M. The manipulation space M as defined in the context of the present invention is circumscribed in the space of work T. The manipulation space is therefore less than or equal to the work space T. Thus, the constraint module 16 makes it possible to limit the positions which can be occupied by the free end 26 of the haptic arm 14 to those located within this manipulation space M.

According to the embodiment shown in FIG. 1, the base 12 and the stress module 16 are offset transversely along the arrangement axis X. This gives the console 10 a stepped profile (see FIG. 5). This offset optimizes the ergonomics of the console 10 and provides greater comfort to the user. This offset also makes it possible to better adhere to the specific anatomy of a virtual patient on whom the surgical simulation is to be performed. Similarly, the arrangement distance between base 12 and constraint module 16 is variable. The constraint module 16 also has, according to certain embodiments, an inclination relative to the base 12. This inclination can be variable.

Beyond the ergonomic reason, the inclination of the foundation intended to accommodate the body of the robot 20 with the haptic arm 14 and the offset of the base 12 with the constraint module 16 makes it possible to create a larger workspace T, thus optimizing the simulation experience.

As mentioned at the beginning of this description, the console 10 according to the present application is used in kit form. More particularly, it is a surgical training kit comprising:
a surgical training console 10 according to the present invention,
and at least one surgical simulation tool 100.

The simulation operates when the user interacts with the haptic arm 14 by means of a surgical simulation tool 100. In some simulations, a virtual twin of the surgical simulation tool 100 used by the user (and connected to the haptic arm 14) is represented to the user by the display device. It can for example, be a tool simulating a catheter or scissors (see FIGS. 5a and 5b). In other cases, the surgical simulation tool 100 is not viewable by the user through the display device. This case is illustrated in FIG. 3. In this particular case, the surgical simulation tool helps to give the user the illusion that his hand is surrounded by organs and soft tissues (while, let's not forget, the manipulation space is empty). It is therefore a tool allowing the user to directly use his hand to receive the haptic feedback (or signal) provided by the haptic arm 14. In the particular case of the surgical simulation tool 100 illustrated in FIG. 3, once the surgical simulation tool 100 is connected to the free end 26 of the haptic arm 14, the user slides his hand into it. Polyurethane foam prevents the hand from being injured, and a spring ensures that the surgical simulation tool 100 clamps the hand sufficiently so that it remains in place despite movements within the manipulation space.

Regardless of their shape and simulated function, all surgical simulation tools 100 connect to the free end 26 of the haptic arm 14 by a Plug and Play connection. In the present application, the notion of “plug and play” describes a simple action, involving only a limited number of gestures, preferably only one. A “plug and play” connection describes a connection that is made with a single gesture.

The console 10 according to the present application makes it possible to respond to this problem of haptic feedback and constraint on the last three degrees of freedom, the proximal degrees of freedom, without having to use a motorization which would weigh down the haptic arm 14 and make it difficult to manipulate, thus making the simulation much less realistic. Thanks to the constraints module 16, the last degrees of freedom are constrained, directly or indirectly, in a simple and effective manner, without danger or discomfort for the user.

1. A surgical training console, comprising:
a base,
a constraint module arranged at a distance from the base along an arrangement axis X, the constraint module having at least one opening enabling the passage of at least a part of at least one surgical simulation tool intended to be manipulated by a user,
a haptic arm secured to the base, the haptic arm having a free end configured to connect the at least one surgical simulation tool, the haptic arm being movable and making it possible to define a workspace comprising a set of positions that can be occupied by the free end,
a control unit connected to the haptic arm, the control unit being configured to generate a virtual reality and define parameters thereof so as to obtain, for the user, a simulation surgical intervention, and
a display device connected to the control unit configured to display, to the user, a representation of a manipulation of the surgical simulation tool in the virtual reality generated by the control unit,
wherein
the parameters of the virtual reality comprise parameters for controlling the mobility of the haptic arm, the base and the constraint module are arranged to delimit an empty space defining a manipulation space, the manipulation space being less than or equal to the work space, and
the constraint module enables to limit the positions which can be occupied by the free end of the haptic arm to those located within the manipulation space.

2. The surgical training console according to claim 1, wherein the constraint module forms a tabletop.

3. The surgical training console according to claim 1, wherein the base and the constraint module are offset transversely along the arrangement axis X, so that the console has a stepped profile.

4. The surgical training console according to claim 1, wherein the arrangement distance between the base and the constraint module is variable.

5. The surgical training console according to claim 1, wherein the constraint module presents an inclination relative to the base.

6. The surgical training console according to the claim 5, wherein this inclination is variable.

7. The surgical training console according to claim 1, wherein the base comprises an inclined foundation intended to receive a robot comprising the haptic arm.

8. The surgical training console according to claim 1, wherein the base and the constraint module are secured to each other in a reversible manner.

9. The surgical training console according to claim 1, wherein the at least one opening has movable edges and a variable diameter.

10. The surgical training console according to claim 1, wherein the display device is associated with a mobile calibration tool, and wherein the console comprises a calibration module having an imprint intended to cooperate with the mobile calibration tool.

11. The surgical training console according to claim 10, wherein the calibration module is reversibly attached to the base.

12. The surgical training console according to claim 1, wherein the display device is a virtual reality headset.

13. The surgical training console according to claim 1, wherein the haptic arm presents a resting position in which the free end is positioned facing the opening of the constraint module.

14. A surgical training kit comprising:
the surgical training console of claim 1; and
at least one surgical simulation tool.

15. The surgical training kit according to claim 14, wherein a virtual twin of the surgical simulation tool connected to the haptic arm is represented to the user by the display device.
